(19) United States
(12) Patent Application Publication
(10) Pub. No.: US 2023/0414114 A1
(43) Pub. Date: Dec. 28, 2023

(54) DISEASE DETECTION SYSTEM

(71) Applicant: VALEO SYSTEMES THERMIQUES, La Verriere (FR)

(72) Inventor: Georges DE PELSEMAEKER, La Verriere (FR)

(73) Assignee: VALEO SYSTEMES THERMIQUES, La Verriere (FR)

(21) Appl. No.: 18/252,269

(22) PCT Filed: Oct. 26, 2021
(86) PCT No.: PCT/EP2021/079597

§ 371 Date: May 9, 2023

(30) Foreign Application Priority Data
Nov. 9, 2020 (FR) 2011469

(51) Int. Cl. A61B 5/0205; A61B 5/05; A61B 5/01; A61B 5/00; G16H 10/60

(52) U.S. Cl. CPC A61B 5/02055; A61B 5/05; A61B 5/01; A61B 5/02433; G16H 10/60; A61B 2560/0252; A61B 5/6893

(57) ABSTRACT

A system for detecting a disease in a person, having an acquisition device configured to acquire examination data on the person, and a data processing device arranged to receive the examination data and including a correction table with at least one corrective term corresponding to a measurement error or to variation of a physical characteristic between individuals; the data processing device being arranged to correct the examination data using the at least one corrective term and consequently generate a corrected value, the data processing device including a memory of reference values and being configured to compare the reference values against the corrected value.

### TECHNICAL FIELD

The present invention relates to a system for detecting a disease in a person, notably a contagious disease such as an acute respiratory distress disease like COVID-19.

### BACKGROUND OF THE INVENTION

Potential COVID-19 patients are currently detected by taking their temperature, by supplementary clinical examinations, then by a COVID-19 test, which still possesses a relatively low confidence level. A pulmonary scan can confirm the seriousness of the disease with a high level of precision.

There is a great need to rapidly detect, notably by virtue of mobile means, potential patients with a contagious disease, for example the disease linked to COVID-19.

### SUMMARY OF THE INVENTION

One subject of the invention is thus a system for detecting a disease in a person, notably a contagious disease such as COVID-19, this detection system comprising:
an acquisition device for acquiring examination data on the person, this acquisition device notably comprising at least one physiological measurement sensor such as a radar, and a thermal camera for acquiring these examination data,
a data processing device arranged to receive these examination data obtained by the acquisition device,
a display device arranged to display diagnostic information for diagnosing the disease based on an analysis of said examination data, it being possible for this diagnostic information to be representative of a level of probability that the person is infected with the disease.

According to one of the aspects of the invention, the data processing device comprises an artificial intelligence unit arranged to process the examination data obtained by the acquisition device and provide said diagnostic information.

According to one of the aspects of the invention, the data processing device and the display device form part of the same apparatus, for example a computer, notably a portable computer.

In this case, the display device is a screen of the portable computer and the data processing device comprises a microprocessor of this computer.

In a variant, the display device is arranged to be visible to the person examined, and notably the display device, which notably comprises a screen, is remote from the data processing device, these display and data processing devices being, for example, connected to one another by a wireless connection, for example by a 3G, 4G or 5G communication protocol, or by the Internet or Wi-Fi, for example.

According to one of the aspects of the invention, the display device and possibly also the data processing device are arranged to be installed in a motor vehicle.

As a variant, the display device and possibly also the data processing device are arranged to be placed in a fixed manner, in a building or an outer courtyard, for example.

According to one of the aspects of the invention, the processing device is arranged to make the diagnosis automatically, without human intervention.

As a result of automatic processing, the invention permits rapid diagnosis and/or mass screening, making it possible to return to work or to lift a lockdown more rapidly.

According to one of the aspects of the invention, the acquisition device for acquiring examination data is arranged to make possible contactless measurements, at a safe distance, of vital signs and thermal and visible imaging on the person to be examined.

The system according to the invention thus advantageously uses the amalgamation of contactless measurements, at a safe distance, of vital signs, and of thermal and visible imaging.

According to one of the aspects of the invention, the artificial intelligence unit is arranged to use a diagnostic model based on the artificial intelligence and fed by a reasonable number of clinical measurements.

As a result of relatively light equipment, the invention notably makes possible facilitated deployment of field hospitals in order to support populations.

The invention makes it possible to perform diagnoses in a mobile manner. It is easy to put in place, for example using a thermal camera, a physiological measurement sensor such as a radar and a laptop personal computer. The invention makes it possible for it to be set up rapidly over a whole area.

According to one of the aspects of the invention, the acquisition device comprises a radar for acquiring data relating to the vital signs of the person, a thermal camera for temperature measurements providing temperature data, and a camera operating in the visible spectrum for characterizing the person tested, providing data for characterizing the person.

According to one of the aspects of the invention, the data processing device is arranged to run a diagnostic algorithm based on an amalgamation of vital data, notably respiratory rate, respiration amplitude, inhalation and exhalation time, heart rate and arrhythmia.

According to one of the aspects of the invention, the algorithm uses temperatures measured in noteworthy regions, which are located by image processing, or indeed the oxygen level linked to the person examined.

Preferably, these noteworthy regions for temperature measurement are located inside the mouth, on the tip of the nose, on the cheeks and the palm of the hand.

According to one of the aspects of the invention, the diagnostic algorithm uses a characterization of the person, such as data relating to age, gender, clothing, height and body mass index (BMI).

According to one of the aspects of the invention, the system is arranged to acquire examination data until the diagnostic information is made available within a time period notably of between 30 and 120 seconds.

According to one of the aspects of the invention, the system is arranged to make it possible to acquire the examination data by making measurements at a distance of 60 cm to 2 m between the acquisition device and the person. It avoids the person having to be in contact with the acquisition device.

The invention thus makes possible a rapid diagnosis, without additional time being needed for referral to a doctor, for example. The diagnostic information can, where applicable, be sent automatically to a doctor and can be held on a data storage system of the Cloud type.

According to one of the aspects of the invention, the data processing device uses an algorithm for analyzing the examination data acquired and, where applicable, for selecting from people with the aim of detecting the cases of diseased persons, based on all of the data collected and an artificial intelligence, the first level of training of which is performed on a sample in a hospital setting.

This training of the artificial intelligence can be performed by means of a set of measurements which is collected by the system, but also by means of medical monitoring of the patients. This permits improvement of the model over time.

According to one of the aspects of the invention, the measurements performed by the acquisition device can serve to subsequently refine the diagnosis made by the artificial intelligence.

By virtue of the invention, a diagnosis can be made without contact with the person, thereby limiting the risks of contamination, this being particularly advantageous, for example, in the case of a pandemic such as that linked to COVID-19.

According to one of the aspects of the invention, the examination data measured comprise at least one of the following data: temperatures measured at different points of the body of the person to be examined, a respiratory or a cardiac characteristic.

According to one of the aspects of the invention, the acquisition device is arranged to acquire examination data comprising an external temperature, a temperature measured on a cheek of the person, a temperature measured on the tip of the nose of the person, and also, where applicable, a maximum temperature of the face and a temperature of an item of clothing or of a surface at a controlled reference temperature.

According to one of the aspects of the invention, the noteworthy measurement points are located by an artificial intelligence by means of an object identification flowchart.

According to one of the aspects of the invention, the temperature relating to a noteworthy point is obtained by averaging over time and by averaging the temperatures of a surface defined by pixels originating from an image from an infrared camera in proximity to the noteworthy point identified on the visible image by means of an object identification algorithm.

According to one of the aspects of the invention, the personal characterizations are identified by means of the red green blue camera (or RGB camera) or far infrared camera (FIR camera) in addition to reading the identity of the person, by virtue of a classification system which can be trained on RGB (red green blue) or infrared images. Using a larger number of parameters, notably age, gender, height, body mass index and phenotype, for example, serves to improve the diagnostic models.

According to one of the aspects of the invention, the diagnostic model, or diagnostic algorithm, which is fed with more data, such as noteworthy temperatures of the body, an ambient temperature, a class of personal characteristics and a time of day, can be arranged to use, in addition, data on the movements of the person examined in order to check whether they have come into contact with a diseased person or have passed through an at-risk area.

According to one of the aspects of the invention, the system is arranged to operate in the absence of a radar and by using the RGB cameras in order to estimate the cardiac and respiratory parameters.

According to one of the aspects of the invention, the temperature relating to a noteworthy point can be obtained by averaging over time and by averaging the temperatures of a surface defined by pixels originating from an image from a camera in proximity to the noteworthy point. The noteworthy point is, for example, defined geometrically by means of an image area called a building box, which surrounds it, for example by means of the geometrical average of the sides of the area of the image. This image area is a surface delimited by a series of points which is constructed by an object identification algorithm.

According to one of the aspects of the invention, the system lacks an RGB camera and/or does not use the surface the temperature of which is controlled. In this case, the system uses the external temperature and a model of heat transfer on the clothed areas or indeed only the differences in temperature between the noteworthy points.

According to one of the aspects of the invention, the system is arranged to use an amalgamation of contactless measurements, notably of vital signs, and of thermal and visible imaging.

The diagnostic information comprises a class chosen from among three predetermined classes, which are “Healthy person”, “Person suspected to have the disease” and “Person highly likely to have the disease”.

The diagnostic information can also comprise an evaluation of the severity of the disease.

Another subject of the invention is a method for providing diagnostic information for detecting a disease in a person, notably a contagious disease such as COVID-19, this method comprising the following steps:
acquiring examination data on the person, using an acquisition device notably comprising at least one physiological measurement sensor such as a radar, and a thermal camera for acquiring these examination data,
receiving these examination data obtained by the acquisition device,
processing these examination data in order to obtain diagnostic information for diagnosing the disease based on an analysis of said examination data,
displaying diagnostic information for diagnosing the disease based on an analysis of said examination data, it being possible for this diagnostic information to be representative of a level of probability that the person is infected with the disease.

The present invention can make checks possible in the public realm in general, notably on lanes of traffic, at entrances and exits of buildings, at airport gates and in schools.

The present invention also permits medical monitoring of people, for example of diseased persons receiving home care.

Another subject of the invention is detection of a disease in a person, notably a contagious disease such as COVID-19, this detection system comprising:
an acquisition device for acquiring examination data on the person, this acquisition device notably comprising at least one physiological measurement sensor such as a radar, and a thermal camera for acquiring these examination data,
a data processing device arranged to receive these examination data obtained by the acquisition device,
this data processing device being arranged to generate a corrected value, also referred to as a standardized value, obtained from at least one examination datum measured by the acquisition device and corrected by a corrective term from a correction table.

By virtue of the invention, it is possible to carry out measurements for example by means of a camera, thermocouples, a radar, an oximeter, which measurements are processed in order to standardize them with a view to obtaining said standardized value and making them comparable against reference data.

The invention makes it possible in particular to correct errors in context measurements and variations between individuals, variations associated with their physical characteristics such as age, gender and/or body mass.

According to one of the aspects of the invention, the standardized value can comprise a component encoded by default or can be a value encoded by default.

According to one of the aspects of the invention, a first corrective term is linked to two effects, a first effect being the correction linked to the effect of the variation in the temperature of the room on the camera of the acquisition device, in particular on the cell of the camera. A drift of the measurement is observed when the temperature of the camera changes. This is the case when the temperature of the room changes because, in general, the measurement is not absolute but differential. The second part of this correction depends on the impact of the ambient temperature on the physiology of the person being tested. When the room temperature rises, the temperature of the person's skin changes slightly in order to keep the body within ideal survival conditions.

According to one of the aspects of the invention, a second corrective term corresponds to a change of reference of the zone of the person to be tested.

According to one aspect of the invention, the third term corresponds to a correction linked to the age of the person tested.

According to one aspect of the invention, the fourth corrective term corresponds to a correction linked to the gender of the person.

According to one of the aspects of the invention, the fifth corrective term corresponds to a level of clothing of the person tested

According to one of the aspects of the invention, the sixth corrective term corresponds to the observation that a person's temperature fluctuates naturally depending on the time of day. This is the circadian cycle. The temperature of the person is standardized at a reference time, for example 6 o'clock in the morning.

According to one of the aspects of the invention, other corrective terms depend respectively on the body mass index (BMI) and the fitness of the person.

According to one of the aspects of the invention, the processing device is arranged to process examination data obtained for example using a camera, thermocouples, a radar, an oximeter of the acquisition device, in order to standardize them with a view to obtaining said standardized value and making them comparable against one or more reference values.

Preferably, a reference value corresponds to a value obtained using synthetic characteristics of a fictitious reference person. These characteristics are preferably average values, measured in particular on a reference population, for example women aged 25 to 35 years old, with a BMI of 25, living in Northern Europe. A fictitious reference person of this kind is called a Persona.

According to one of the aspects of the invention, the acquisition device comprises a radar for acquiring at least one datum relating to a vital sign of a person, two thermal cameras for acquiring temperature data, and a camera operating in the visible light range to acquire a characterization datum of a tested person.

According to one of the aspects of the invention, the acquisition device further comprises an NIR camera, capable of operating in the near infrared, with an illuminator, this camera being arranged to acquire an ambient temperature datum, ambient light datum and time of day datum.

According to one of the aspects of the invention, the acquisition device also comprises an NIR camera, capable of operating in the near infrared, arranged to perform an oximetry measurement and measure the respiratory characteristics of the person tested.

According to one of the aspects of the invention, the examination data acquired by the acquisition device are chosen from among: a characteristic of a temperature relative to a person tested, a cardiac characteristic, a respiratory characteristic or a blood circulation characteristic of the person tested.

According to one of the aspects of the invention, the processing device comprises a memory of reference values against which the corrected values resulting from the examination data acquired by the acquisition device are compared.

According to one of the aspects of the invention, the corrected values have undergone a correction based on taking account of errors in context measurement and/or variations between individuals, variations associated with their physical characteristics such as age, gender and body mass.

According to one of the aspects of the invention, these cameras and radar are in particular installed in a vehicle.

According to one of the aspects of the invention, the data processing device is arranged to carry out an amalgamation of data resulting from the acquisition of data from the aforementioned camera and radar, these data notably comprising data relating to vital signs, temperature data and characterization data of the person tested, ambient temperature data, ambient light data and time of day data.

According to one of the aspects of the invention, the data processing device is arranged to run a diagnostic algorithm, based on an amalgamation of vital data including, notably respiratory rate, respiratory amplitude, inhalation and exhalation time, heart rate and arrhythmia.

According to one of the aspects of the invention, the amalgamation of data to perform the diagnosis also includes data from an indirect oximetry measurement, temperature data measured on specific regions of the person tested, regions located in particular by image processing, these regions being for example the inside of the mouth, the tip of the nose, the cheekbones and/or the palm of the hand of the person tested, a characterization datum of the person such as age, gender, clothing, body mass index (BMI), genotype, and environmental data such as ambient temperature.

According to one of the aspects of the invention, the system comprises a display device arranged to display diagnostic information for diagnosing the disease based on an analysis of said examination data, it being possible for this diagnostic information to be representative of a level of probability that the person is infected with the disease.

The oximetry datum of the person tested is in particular a function of a relative variation of the values obtained for example by the two NIR and FIR cameras between two lighting states with the illuminator and identifying the predefined or learned noteworthy points thanks to the camera operating in the visible light domain and an object identification model defined by learning. For the processing device, this entails placing a virtual mesh on the face of the person tested and following the nodes which define this mesh. The two NIR and FIR cameras produce different images which vary when the wavelengths of the incident light change and a fortiori when the illuminator emits different non-visible artificial lights. The comparison of the values read on the FIR and NIR cameras with and without illumination makes it possible to identify the oxygenation of the person being tested. To improve accuracy, the illumination can be at multiple wavelengths, because the absorption and the reflection from the test person's skin depends on the test person's oxygenation level or red blood cell components. The invention thus proposes in particular to compare the values between the different points of the face in order to complete this evaluation. This comparison also makes it possible to assess circulation problems and potentially skin problems. The resolution of the facial mesh can be wide or finer depending on the expected accuracy or the diagnosis to be made.

The invention thus proposes addition of the evaluation of oxygenation in the blood for comparative measurement between thermal cameras in the presence or absence of one or more illuminations with different or centered spectra on different wavelengths, or by identification of the metabolism by studying the difference in behavior of several distinctive points (nose, cheekbone, hand, forehead, mouth, eye, etc.).

According to one of the aspects of the invention, the processing device is arranged to carry out a correction by taking into account the circadian cycle and the ambient temperature for the threshold values, but also optionally to correct drifts in measurements of the MR and FIR cameras and also of a possible radar.

### BRIEF DESCRIPTION OF DRAWINGS

The invention and its various applications will be better understood upon reading the following description and examining the accompanying figures, in which:

FIG. 1 schematically illustrates a system according to a non-limiting embodiment of the invention;

FIG. 2 illustrates a block diagram illustrating the steps implemented in the system of FIG. 1; and

FIG. 3 illustrates a table with various corrective terms used in a system according to an exemplary embodiment of the invention.

### DETAILED DESCRIPTION OF THE INVENTION

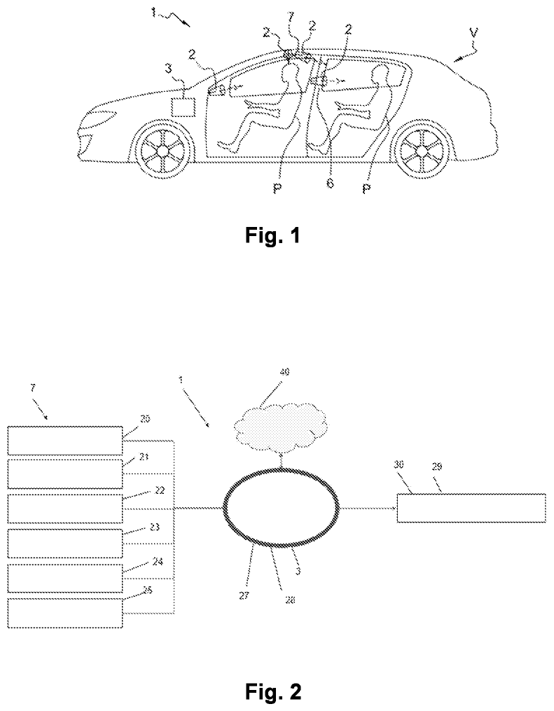

FIGS. 1 and 2 show a system 1 for detecting a disease in a person, notably a contagious disease such as COVID-19, according to the invention.

This system 1 comprises:
an acquisition device 7 for acquiring examination data on the person,
a data processing device 3 arranged to receive these examination data obtained by the acquisition device 7,
a display device 30 arranged to display diagnostic information for diagnosing the disease based on an analysis of said examination data, it being possible for this diagnostic information to be representative of a level of probability that the person is infected with the disease.

This system 1 comprises in particular:
a sensor for sensing the cardiac activity of at least one passenger, in this instance the heart rate, this sensor being a camera operating in the near infrared,
a sensor for sensing the respiratory activity, notably in terms of respiratory amplitude and/or frequency, of at least one passenger, this sensor being a camera operating in the far infrared, or thermal camera,
a radar arranged to measure vital signs of the person,
a sensor for sensing the profile characteristics of the passenger, notably their gender, weight, height and age, this sensor being in this instance a red green blue camera, also called an RGB camera,
a card reader for reading an identity card of the person and obtaining personal data of the person.

These sensors and cameras, which form part of the acquisition device 7, are shown by the reference sign 2 in FIG. 1. Certain sensors 2 are, for example, positioned on a ceiling of the vehicle. One of the other cameras 2 is positioned in a side pillar 6 of the vehicle V.

The heart rate and respiration sensor can be in the back of the seat or in the central console level with the thigh of the passenger, this being non-limiting.

These sensors 2 are connected in order to exchange information with a data processing device 3 placed on the vehicle V.

The data processing device 3 comprises an artificial intelligence unit arranged to process the examination data obtained by the acquisition device 7 and provide said diagnostic information.

The data processing device 3 and the display device 30 form part of the same apparatus, for example a computer, notably a portable computer.

In this case, the display device 30 is a screen of the portable computer and the data processing device comprises a microprocessor of this computer.

The display device 30 is arranged to be visible to the person examined, and notably the display device, which notably comprises a screen, is remote from the data processing device, these display and data processing devices being, for example, connected to one another by a wireless connection, for example by a 3G, 4G or 5G communication protocol, or by the Internet or Wi-Fi, for example.

The display device 30 and also the data processing device 3 are in this instance arranged to be installed in a motor vehicle.

As a variant, the display device 30 and possibly also the data processing device 3 are arranged to be placed in a fixed manner, in a building or an outer courtyard, for example.

The processing device 3 is arranged to make the diagnosis automatically, without human intervention.

The acquisition device 7 for acquiring examination data is arranged to permit contactless measurements, at a safe distance, of vital signs and thermal and visible imaging on the person to be examined.

The system according to the invention thus advantageously uses the amalgamation of contactless measurements, at a safe distance, of vital signs and thermal and visible imaging.

The artificial intelligence unit is arranged to use a diagnostic model based on an artificial intelligence and fed by a reasonable number of clinical measurements.

The invention makes it possible to perform diagnoses in a mobile manner. It is easy to set up, for example using a thermal camera, a physiological measurement sensor such as a radar and a laptop personal computer.

The acquisition device 7 comprises a radar for acquiring data relating to the vital signs of the person, a thermal camera for temperature measurements providing temperature data, and a camera operating in the visible spectrum for characterizing the person tested, providing data for characterizing the person.

The data processing device 3 is arranged to run a diagnostic algorithm based on an amalgamation of vital data, notably respiratory rate, respiration amplitude, inhalation and exhalation time, heart rate and arrhythmia and oximetry.

The algorithm uses temperatures measured in noteworthy areas, located by processing the image.

Preferably, these noteworthy areas for temperature measurement are located inside the mouth, on the tip of the nose, on the cheeks and the palm of the hand.

The diagnostic algorithm uses a characterization of the person, such as age, gender, clothing, height and body mass index data.

The system is arranged to acquire examination data until the diagnostic information is made available within a time period notably of between 30 and 120 seconds.

The invention thus permits a rapid diagnosis without additional time for being referred to a doctor, for example. The diagnostic information can, where applicable, be sent automatically to a doctor and can be held on a data storage system 40 of the Cloud type.

The data processing device 3 uses an algorithm for analyzing the examination data acquired and, where applicable, for sorting through people with the aim of detecting the cases of diseased people, based on all of the data collected and an artificial intelligence, the first level of training of which is performed on a sample in a hospital setting.

The measurements performed by the acquisition device can serve to subsequently refine the diagnosis made by the artificial intelligence.

By virtue of the invention, a diagnosis can be made without contact with the person, thereby limiting the risks of contamination, this being particularly advantageous, for example, in the case of a pandemic such as that linked to COVID-19.

According to one of the aspects of the invention, the examination data measured comprise at least one of the following data: temperatures measured at different points of the body of the person to be examined, a respiratory or a cardiac characteristic.

The acquisition device 7 is arranged to acquire examination data comprising an external temperature, a temperature measured on a cheek of the person, a temperature measured on the tip of the nose of the person, and also, where applicable, a maximum temperature of the face and a temperature of an item of clothing or of a surface at a controlled reference temperature.

The noteworthy measurement points are located by an artificial intelligence by means of an object identification flowchart.

The personal characterizations are identified by means of the red green blue camera (or RGB camera) or far infrared camera (FIR camera) in addition to reading the identity of the person, by virtue of a classification system which can be trained on RGB (red green blue) or infrared images. Using a larger number of parameters, notably age, gender, height, body mass index and phenotype, for example, serves to improve the diagnostic models.

The diagnostic model, or diagnostic algorithm, which is fed with more data, such as noteworthy temperatures of the body, an ambient temperature, a class of personal characteristics and a time of day, can be arranged to use, in addition, data on the movements of the person examined in order to check whether they have come into contact with a diseased person or have passed through an at-risk area.

The temperature relating to a noteworthy point can be obtained by averaging over time and by averaging the temperatures of a surface defined by pixels originating from an image from a camera in proximity to the noteworthy point. The noteworthy point is, for example, defined geometrically by means of an image area called a building box, which surrounds it. This image area is a surface delimited by a series of points which is constructed by an object identification algorithm.

The diagnostic information comprises a class chosen from among three predetermined classes, which are “Healthy person”, “Person suspected to have the disease” and “Person highly likely to have the disease”.

The invention thus implements the following steps:
acquiring examination data on the person using the acquisition device 7, which are the steps 20 to 25 of FIG. 2,
receiving these examination data obtained by the acquisition device (step 27),
processing these examination data in order to obtain diagnostic information for diagnosing the disease based on an analysis of said examination data, using the data processing device 3 (step 28),
displaying diagnostic information for diagnosing the disease based on an analysis of said examination data, it being possible for this diagnostic information to be representative of a level of probability that the person is infected with the disease (step 29).

The steps 20 to 25 are the following:
identifying the personal characterizations by means of the red green blue cameras (or RGB cameras), which is step 20,
acquiring the temperature of the person in step 21 with a thermal camera of FIR type, the details of this temperature acquisition having already been described,
acquiring the respiratory rate in step 22 using the FIR camera,
acquiring the heart rate in step 23 using the camera in the near infrared, or NIR camera, namely a camera capable of operating in the near infrared,
acquiring vital signs using the radar, in step 24,
acquiring personal data using the card reader in step 25,
potentially oximetry by the near infrared (NIR) camera.

The present invention also permits medical monitoring of people.

The examination data can, where applicable, comprise the size of the pupils and their position.

The diagnostic information is sent automatically to a remote data storage system of the Cloud type.

Likewise, the information which is useful to the diagnostic model, or diagnostic algorithm, can be received from the remote system of the Cloud type.

Potentially two cameras, NIR and FIR, are used. The FIR camera for the temperatures and the characteristics of the respiration and the NIR camera for the oximetry and the heart rate.

The acquisition device is notably arranged to acquire examination data comprising an external temperature, a temperature measured on a cheek of the person, a temperature measured on the tip of the nose of the person, and also, where applicable, a maximum temperature of the face and a temperature of an item of clothing or of a surface at a controlled reference temperature, and, where applicable, a tidal volume, tremors and the blood oxygen level.

We will now describe a variant of the invention which makes it possible to improve the processing of the data and to have a more reliable diagnosis.

The detection system 1 comprises:
an acquisition device 7 for acquiring examination data on the person, this acquisition device notably comprising at least one physiological measurement sensor such as a radar, and a thermal camera for acquiring these examination data,
a data processing device 3 arranged to receive these examination data obtained by the acquisition device,
this data processing device 3 being arranged to generate a corrected value, also called a standardized value, obtained from at least one examination datum measured by the acquisition device 7 and corrected by a corrective term from a correction table.

This correction table can be a table stored by the processing device or, as a variant, a table of values that is updated, notably by virtue of an artificial intelligence unit.

The processing device is arranged to process examination data obtained for example using a camera, thermocouples, a radar, an oximeter of the acquisition device, in order to standardize them with a view to obtaining said standardized value and making them comparable against one or more reference values.

In the example described, the acquisition device 7 comprises a radar for acquiring at least one datum relating to a vital sign of a person, two thermal cameras for acquiring temperature data, and a camera operating in the visible light range to acquire a characterization datum of a tested person.

The acquisition device 7 further comprises an NIR camera, capable of operating in the near infrared, with an illuminator, this camera being arranged to acquire an ambient temperature datum, ambient light datum and time of day datum.

The acquisition device 7 also includes an NIR camera, capable of operating in the near infrared, arranged to perform an oximetry measurement and measure the respiratory characteristics of the person tested.

The examination data acquired by the acquisition device are chosen from among: a characteristic of a temperature relative to a person tested, a cardiac characteristic, a respiratory characteristic or a blood circulation characteristic of the person tested.

The processing device comprises a memory of reference values against which the corrected values resulting from the examination data acquired by the acquisition device are compared.

The corrected values have undergone a correction based on taking account of errors in context measurement and/or variations between individuals, variations associated with their physical characteristics such as age, gender and body mass.

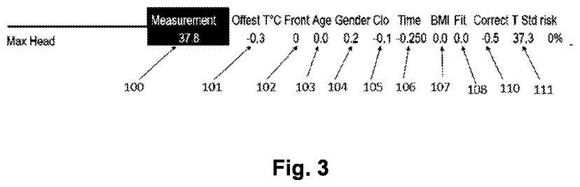

For example, with reference to the table in FIG. 3, the temperature taken on the head of the person tested using the acquisition device 7 is 37.8°, which is a temperature usually considered to be that of a person with a fever. This measurement corresponds to an examination datum 100.

The invention here makes it possible to correct this conclusion “person with a fever”/“person without a fever” according to corrective terms of the correction table.

We will illustrate this point here.

Let us consider a person tested who is located in a room.

The first correction, associated with a corrective term 101, is linked to two effects. A first effect is the correction linked to the effect of the variation in the temperature of the room on the camera of the acquisition device 7, in particular on the cell of the camera. A drift of the measurement is observed when the temperature of the camera changes. This is the case when the temperature of the room changes because, in general, the measurement is not absolute but differential. The second part of this correction depends on the impact of the ambient temperature on the physiology of the person being tested. When the room temperature rises, the temperature of the person's skin changes slightly in order to keep the body within ideal survival conditions. In the example described here, the sum of the two correction effects gives a corrective term of −0.3° C. because the reference temperature is 20° and the room temperature here is 23° C.

For example, a body temperature of the person tested of 37.5° C. at 23° C. ambient corresponds respectively to 37.2° C. at 20° C.

The second term, reference 102, has a value of equal to 0. This term 102 is chosen, in the context of a change of reference, to bring the temperature measured at the level of the mouth to that of the forehead. For example, 37.5° C. in the mouth corresponds to 37.8° C. on the forehead.

The third term, reference 103, corresponds to a correction linked to the age of the person tested, here of value 0. In other cases, a corrective term of 0.1° C. is chosen for a patient of 50 years compared to a reference of 30 years. Thus, a measurement of 37.5° C. at 50 years old corresponds to 37.6° C. at 30 years old.

The fourth corrective term, reference 104, corresponds to a correction related to gender. For example, women have a slightly higher average temperature than men. The standardized temperature is therefore calculated with respect to the female gender, and the corrective term 104 is applied for a tested person who is male.

The fifth corrective term, reference 105, corresponds to a level of clothing of the person tested.

The sixth corrective term, reference 106, corresponds to the observation that a person's temperature fluctuates naturally depending on the time of day. This is the circadian cycle. The temperature of the person is standardized at a reference time, for example 6 o'clock in the morning.

The seventh and eighth corrective terms 107 and 108 depend respectively on the body mass index (BMI) and the fitness of the person.

The global correction, represented by the global corrective term 110, is the sum of the corrective terms 101 to 108. This global corrective term 110 permits standardization of the temperature measurement, which allows a comparison with one or more threshold values and therefore makes it possible to define a risk of contamination. Here, the corrected temperature 111 is 37.3°, which corresponds to a 0% risk of having a fever.

Thus, the corrected temperature, here 37.3°, is compared against a stored reference temperature, here a fever threshold value at 37.7°, for example.

The same methodology can be used for cardiac, respiratory and circulatory characteristics and can thus define a set of standardized measurements that can be used for diagnosis.

In the example described, the data processing device 3 is arranged to run a diagnostic algorithm, based on an amalgamation of vital data including notably respiratory rate, respiratory amplitude, inhalation and exhalation time, heart rate and arrhythmia. All of these vital data can undergo standardization in the manner described above using corrective terms.

The corrected values are compared against reference values, in particular threshold values, in order to conclude on diagnostic information.

What is claimed is:

1. A system for detecting a disease in a person, comprising:
an acquisition device configured to acquire examination data on the person, and
a data processing device arranged to receive the examination data, with the data processing device including a correction table with at least one corrective term corresponding to a measurement error or to variation of a physical characteristic between individuals;
the data processing device being configured to correct the examination data using the at least one corrective term and consequently generate a corrected value,
the data processing device including a memory of reference values and being configured to compare the reference values against the corrected value.

2. The system as claimed in claim 1, wherein the data processing device is configured to process the examination data so that it is comparable against one or more reference values.

3. The system as claimed in claim 1, wherein the acquisition device includes a radar for acquiring examination data relating to a vital sign of the person, a thermal camera for acquiring examination data representative of temperature, and a camera configured to operate in the visible light range.

4. The system as claimed in claim 1, wherein the acquisition device includes an NIR camera with an illuminator, capable of operating in the near infrared, and arranged to acquire examination data representative of ambient temperature, ambient light and time of day.

5. The system as claimed in claim 1, wherein the examination data includes representation of characteristics of the person chosen from among: temperature characteristic, a cardiac characteristic, a respiratory characteristic, and a blood circulation characteristic.

6. (canceled)

7. The system as claimed in claim 1, wherein the correction table includes corrective terms corresponding to measurement errors and to variations between persons, associated with their characteristics selected from a group of: age, gender, and body mass.

8. The system as claimed in claim 3, wherein the data processing device is arranged to carry out an amalgamation of data resulting from the acquisition of the examination data from the camera and the radar.

9. The system as claimed in claim 1, wherein the acquisition device includes a NIR camera, a FIR camera and a radar, the data processing device being configured to carry out a correction by taking into account, as threshold values, the circadian cycle and the ambient temperature, while correcting drifts in measurements of the NIR and FIR cameras and of the radar.
